(19) United States
(12) Patent Application Publication
(10) Pub. No.: US 2024/0209036 A1
(43) Pub. Date: Jun. 27, 2024

(54) PEPTIDES AND METHODS OF USE

(71) Applicant: REALTA LIFE SCIENCES, INC., Norfolk, VA (US)

(72) Inventors: Neel K. KRISHNA, Norfolk, VA (US); Kenji CUNNION, Norfolk, VA (US); Ulrich THIENEL, Norfolk, VA (US)

(73) Assignee: REALTA LIFE SCIENCES, INC., Norfolk, VA (US)

(21) Appl. No.: 18/288,274

(22) PCT Filed: May 31, 2022
(86) PCT No.: PCT/US2022/031559

§ 371 Date: Oct. 25, 2023

Related U.S. Application Data
(60) Provisional application No. 63/279,423, filed on Nov. 15, 2021.
(60) Provisional application No. 63/195,401, filed on Jun. 1, 2021.

(51) Int. Cl. C07K 14/08; A61K 45/06; A61P 43/00; A61K 38/00

(52) U.S. Cl. CPC C07K 14/08; A61K 45/06; A61P 43/00; A61K 38/00; C12N 2770/12022

(57) ABSTRACT

The present invention provides peptides that are synthetic modifications of Polar Assortant (PA) peptide including C-terminal PEGylation. The invention further provides methods of using least one synthetic peptide for regulating the complement system and interacting with neutrophils to alter their binding and activity.

### CROSS-REFERENCE TO RELATED APPLICATIONS

This application claims priority to U.S. Provisional Application No. 63/279,423, filed Nov. 15, 2021, and U.S. Provisional Application No. 63/195,401, filed on Jun. 1, 2021. The disclosures of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on May 25, 2022, is named 251110_000164_SL.txt and is 1,196 bytes in size.

### BACKGROUND OF THE INVENTION

Embodiments of the present invention relates generally to synthetic peptides and uses thereof for therapy, and more specifically to modifications of the synthetic peptides that can prevent, treat and/or mitigate toxicity arising from checkpoint inhibitors, particularly intestinal necrosis or damage.

### The Complement System

The complement system, an essential component of the innate immune system, plays a critical role as a defense mechanism against invading pathogens, primes adaptive immune responses, and helps remove immune complexes and apoptotic cells. Three different pathways comprise the complement system: the classical pathway, the lectin pathway and alternative pathway. C1q and mannose-binding lectin (MBL) are the structurally related recognition molecules of the classical and lectin pathways, respectively. Whereas IgM or clustered IgG serve as the principal ligands for C1q, MBL recognizes polysaccharides such as mannan. Ligand binding by C1q and MBL results in the sequential activation of C4 and C2 to form the classical and lectin pathway C3-convertase, respectively. In contrast, alternative pathway activation does not require a recognition molecule, but can amplify C3 activation initiated by the classical or lectin pathways. Activation of any of these three pathways results in the formation of inflammatory mediators (C3a and C5a) and the membrane attack complex (MAC), which causes cellular lysis.

While the complement system plays a critical role in many protective immune functions, complement activation is a significant mediator of tissue damage in a wide range of autoimmune and inflammatory disease processes. (Ricklin and Lambris, “Complement-targeted therapeutics.” Nat Biotechnol 2007; 25(11):1265-75).

A need exists for complement regulators. On the one hand, the complement system is a vital host defense against pathogenic organisms. On the other hand, its unchecked activation can cause devastating host cell damage. Currently, despite the known morbidity and mortality associated with complement dysregulation in many disease processes, including autoimmune diseases such as systemic lupus erythematosus, myasthenia gravis, and multiple sclerosis, only two anti-complement therapies have recently been approved for use in humans: 1) eculizumab (Soliris™) and 2) ultomiris (Ravulizumab™)two humanized, long-acting monoclonal antibodies against C5 used in the treatment of paroxysmal nocturnal hemoglobinuria (PNH) and atypical hemolytic uremic syndrome (aHUS). PNH and aHUS are orphan diseases in which very few people are afflicted. Currently, no complement regulators are approved for the more common disease processes in which dysregulated complement activation plays a pivotal role. Dysregulated complement activation can play a role in both chronic disease indications and acute disease indications.

Developing peptides to inhibit classical, lectin and alternative pathways of the complement system is needed, as each of these three pathways have been demonstrated to contribute to numerous autoimmune and inflammatory disease processes. Specific blockade of classical and lectin pathways is particularly needed, as both of these pathways have been implicated in ischemia reperfusion-induced injury and other diseases in many animal models. Humans with alternative pathway deficiencies suffer severe bacterial infections. Thus, a functional alternative pathway is essential for immune surveillance against invading pathogens.

The PIC1 family of molecules (also referred to herein as the EPICC family or EPICC peptides) comprise a collection of rationally designed peptides, based on a scrambled astroviral coat protein, that have several anti-inflammatory functional properties including inhibition of the classical pathway of complement, myeloperoxidase inhibition, neutrophil extracellular trap (NET) inhibition and antioxidant activity. The original compound is a 15 amino acid peptide sequence, IALILEPICCQERAA (SEQ ID NO: 1), with a C-terminal monodisperse 24-mer PEGylated moiety (IALILEPICCQERAA-dPEG24; PA-dPEG24; RLS-0071; SEQ ID NO: 2) increasing its aqueous solubility. PA-dPEG24 is a peptide inhibitor of the classical and lectin pathways as well as myeloperoxidase activity and NETosis, which are major effectors of neutrophils [6-8]. A sarcosine substitution scan of SEQ ID NO: 2 revealed that replacement of isoleucine at position 8 with sarcosine resulted in a peptide, IALILEP(Sar)CCQERAA (PA-I8Sar; RLS-0088; SEQ ID NO: 3) that was water soluble without PEGylation (as described in U.S. Pat. No. 10,005,818).

Additional characteristics of the PA-dPEG24 molecule and the PA-I8Sar molecule are discussed herein.

### Checkpoint Therapeutics and Associated Toxicity

Intestinal necrosis is a potentially life-threatening medical condition that can arise from a variety of causes including bacteria sepsis and systemic inflammatory response syndrome (SIRS). The etiology of intestinal necrosis includes a variety of disease processes that can result in vascular compromise, such as venous thrombosis, chronic ischemia, mechanical obstruction and non-obstructive mesenteric ischemia [1] as well as autoimmune inflammatory bowel diseases, which include Crohn's disease and ulcerative colitis, or toxic megacolon from C. difficile colitis.

Additionally, intestinal necrosis due to severe inflammatory responses is a treatment-limiting severe adverse event associated with cancer checkpoint inhibitor drugs. [2] Intestinal necrosis generally compromises the intestinal lumen, leading to transmigration of enteric bacteria and eventually gross leakage of intestinal contents containing bacteria, toxins and other microbial products into the peritoneum and systemic circulation. This process can subsequently lead to bacterial sepsis, hypotension, disseminated intravascular coagulation, multisystem organ failure, and often death. Primary therapeutic intervention in the treatment of intestinal necrosis includes antibiotics to kill bacterial pathogens in the peritoneum and the bloodstream. The use of anti-complement therapy, such as eculizumab, can result in an immunocompromised subject and carries an increased risk for invasive N. meningitidis infections. Further, persistent neutropenia is associated with a high risk of life-threatening bacterial sepsis. Thus, modulation of the complement system and neutrophil effector functions are of concern for potentially worsening the risk of overwhelming bacterial infection due to intestinal necrosis.

There is a need in the art for peptide-based inhibitors of the different pathways of the complement system. There is also a need in the art for therapeutic peptides to prevent, treat and/or mitigate toxic side effects, particularly intestinal necrosis or damage, associated with checkpoint inhibitors.

### BRIEF SUMMARY OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to synthetic peptides that are PEGylated or contain a sarcosine substitution and their use in methods of regulating the complement system and preventing, treating and/or mitigating toxic side effects associated with checkpoint therapeutics, particularly intestinal necrosis or damage.

In one aspect, the present invention provides synthetic peptides that regulate the complement system and methods of using these peptides. Specifically, in some embodiments, the synthetic peptides can bind, regulate and inactivate C1 and MBL, and therefore can efficiently inhibit classical and lectin pathway activation at its earliest point of the complement cascade while leaving the alternative pathway intact. These peptides are of therapeutic value for selectively regulating and inhibiting C1 and MBL activation without affecting the alternative pathway and can be used for treating diseases mediated by dysregulated activation of the classical and lectin pathways. In other embodiments, the peptides regulate classical pathway activation but not lectin pathway activation. The peptides are useful for various therapeutic indications.

In any embodiment, the synthetic peptides are capable of preventing, treating and/or mitigating toxic side effects of checkpoint inhibitors.

In any embodiment, the synthetic peptides are capable of preventing, treating and/or mitigating intestinal necrosis and/or damage, e.g., necrosis or damage resulting from severe inflammatory responses, intestinal infarction (i.e., ischemia reperfusion injury of intestinal tissue), autoimmune inflammatory bowel disease (IBD) and associated therapies, and chemotherapy-induced or toxin-induced intestinal necrosis.

In any embodiment, the invention is based on the identification and modification of peptides of 15 amino acids from Polar Assortant (PA) peptide (SEQ ID NO: 1), derivatives of the peptides, and methods of their use. The PA peptide is a scrambled peptide derived from human astrovirus protein, called CP1. The PA peptide is also known as PIC1 (Peptide Inhibitors of Complement C1), AstroFend, AF, or SEQ ID NO: 1. The PIC1 peptide was originally named as such because it was found to be associated with diseases mediated by the complement system. A PEGylated form of the PIC1 peptide, called PA-dPEG24 or RLS-0071 (SEQ ID NO: 2), has 24 PEG units on the C terminus of the peptide and was shown to have improved solubility in aqueous solution. A sarcosine substitution scan of SEQ ID NO: 2 revealed that replacement of isoleucine at position 8 with sarcosine resulted in a peptide, IALILEP(Sar)CCQERAA (PA-I8Sar; RLS-0088; SEQ ID NO: 3) that was water soluble without PEGylation (as described in U.S. Pat. No. 10,005,818).

In an aspect, the present invention provides a method of preventing, treating and/or mitigating toxic side effects of checkpoint inhibitors comprising administering to the subject in need thereof a composition comprising a therapeutically effective amount of a synthetic peptide comprising SEQ ID NO: 2 and/or 3.

In an aspect, the present invention provides a method of preventing, treating and/or mitigating intestinal necrosis and/or damage, e.g., necrosis or damage resulting from severe inflammatory responses, intestinal infarction (i.e., ischemia reperfusion injury of intestinal tissue), autoimmune inflammatory bowel disease (IBD) and associated therapies, and chemotherapy-induced or toxin-induced intestinal necrosis, comprising administering to the subject in need thereof a composition comprising a therapeutically effective amount of a synthetic peptide comprising SEQ ID NO: 2 and/or 3.

In an aspect, the present invention provides a method of preventing, treating and/or mitigating intestinal necrosis and/or damage in a subject who is being treated, has been treated, and/or will be treated with at least one checkpoint inhibitor comprising administering to the subject in need thereof a composition comprising a therapeutically effective amount of a synthetic peptide comprising SEQ ID NO: 2 and/or 3.

In an embodiment of any of the foregoing methods, the composition further comprises at least one pharmaceutically acceptable carrier, diluent, stabilizer, or excipient. In an embodiment of any of the foregoing methods, the therapeutically effective amount of SEQ ID NO: 2 and/or 3 is about 10 mg/kg to about 160 mg/kg. In an embodiment of any of the foregoing methods, the therapeutically effective amount of SEQ ID NO: 2 and/or 3 is about 20 mg/kg to about 160 mg/kg. In an embodiment of any of the foregoing methods, the therapeutically effective amount of SEQ ID NO: 2 and/or 3 is about 40 mg/kg to about 160 mg/kg. In any embodiment, the therapeutically effective amount of SEQ ID NO: 2 and/or 3 is administered in at least one dose, the first dose comprising about 1 mg/kg to about 160 mg/kg SEQ ID NO: 2 and/or 3. In any embodiment, a second dose comprising a therapeutically effective amount of SEQ ID NO: 2 and/or 3 is administered, the second dose comprising about 1 mg/kg to about 120 mg/kg SEQ ID NO: 2 and/or 3, including about 10 mg/kg to about 120 mg/kg SEQ ID NO: 2 and/or 3. In any embodiment, the therapeutically effective amount of SEQ ID NO: 2 and/or 3 is administered in two doses, the first dose comprising about 1 mg/kg to about 160 mg/kg SEQ ID NO: 2, including about 10 mg/kg to about 120 mg/kg SEQ ID NO: 2 and/or 3, and the second dose comprising about 1 mg/kg to about 120 mg/kg SEQ ID NO: 2 and/or 3, including about 1 mg/kg to about 40 mg/kg SEQ ID NO: 2 and/or 3. In any embodiment, a second dose is administered 30 seconds to 10 hours after a first dose is administered, including about eight hours after administration of the first dose. In any embodiment, the therapeutically effective amount of SEQ ID NO: 2 and/or 3 is administered in multiple doses over a period of about one week to about two weeks, each dose comprising about 1 mg/kg to about 160 mg/kg SEQ ID NO: 2 and/or 3, including about 1 mg/kg to about 120 mg/kg SEQ ID NO: 2 and/or 3, being administered every 4 to 10 hours, including about every eight hours.

In any embodiment, at least one loading dose of about 10 mg/kg to about 160 mg/kg SEQ ID NO: 2 and/or 3 is administered, including about 10 mg/kg to about 120 mg/kg SEQ ID NO: 2 and/or 3, followed by at least one maintenance dose of about 1 mg/kg to about 120 mg/kg SEQ ID NO: 2 and/or 3, including about 1 mg/kg to about 40 mg/kg SEQ ID NO: 2 and/or 3. In any embodiment, the first maintenance dose is administered 4 to 10 hours after the last loading dose. In any embodiment, the maintenance doses are administered every 4 to 10 hours for a period of about one week to about two weeks. In any embodiment, the first maintenance dose is administered 8 hours after the last loading dose, and the maintenance doses are administered every 8 hours for a period of about one week to about two weeks.

In any embodiment of any of the foregoing methods, the composition is formulated for subcutaneous, intravenous, intraperitoneal, or intramuscular administration. In an embodiment, the composition further comprises a pharmaceutically acceptable carrier and/or excipient.

These and other objects, features and advantages of the present invention will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below. The patent or application file contains at least one drawing executed in color. Copies of this patent or patent application publication with color drawing(s) will be provided by the Office upon request and payment of the necessary fee.

FIG. 1 shows that PA-dPEG24 (also referred to herein as RLS-0071) increases survival of rats after cecal ligation. Kaplan-Meier survival curve assessment. The red line indicates the outcome after 75% CLP in animals not receiving treatment (n=6), whereas red curves represent the outcome after animals received a single dose of 40 mg/kg RLS-0071 (n=9).

FIG. 2 shows that RLS-0071 reduces free DNA levels in the blood. Plasma was isolated before surgery (pre-bleed) (n=7) and from animals subject to CLP with (n=4) and without (n=3) administration of a single dose of RLS-0071 24 hours post-surgery. Plasma samples were incubated with PicoGreen. Fluorescence was read at an excitation wavelength of 485 nm and an emission wavelength of 520 nm in a microplate reader. Data are means and standard error of the means.

FIG. 3 shows that RLS-0071 reduces IL-6 levels in the blood. Plasma was isolated before surgery (pre-bleed) (n=8) and from animals subject to CLP with (n=3) and without (n=5) administration of a single dose of RLS-0071 24 hours post-surgery. Plasma samples were analyzed in an IL-6 ELISA according to the manufacturer's instructions. Data are means and standard error of the means.

FIG. 4 shows that multidose administration of RLS-0071 increases survival of rats after cecal ligation via a Kaplan-Meier survival curve assessment. The black line indicates the outcome after 75% CLP in animals not receiving treatment (n=12), whereas the green line represents the outcome after animals received dosing of 40 mg/kg RLS-0071 at 0.5, 24, 28 and 72-hours post-surgery (n=12).

FIG. 5 shows that multidose administration of RLS-0088 increases survival of rats after cecal ligation via a Kaplan-Meier survival curve assessment. The red line indicates the outcome after 75% CLP in animals receiving saline only (n=3), whereas the green line represents the outcome after animals received doses of 40 mg/kg RLS-0071 at 0.5, 24, 28 and 72-hours post-surgery (n=5). SID=once daily dosing.

### DETAILED DESCRIPTION OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to synthetic peptides that are PEGylated or contain a sarcosine substitution and their use in methods of regulating the complement system and mitigating toxic side effects associated with checkpoint therapeutics, particularly intestinal necrosis or damage.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

As used herein, the term “combination” of a synthetic peptide according to the claimed invention and at least a second pharmaceutically active ingredient means at least two, but any desired combination of compounds can be delivered simultaneously or sequentially (e.g., within a 24 hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “analog” or “functional analog” refers to a related modified form of a polypeptide, wherein at least one amino acid substitution, deletion, or addition has been made such that said analog retains substantially the same biological activity as the unmodified form, in vivo and/or in vitro.

The terms “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

Several different computer programs are available to determine the degree of identity between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at www.accelrys.com/products/gcg), using either a Blosum 62 matrix or a PAM250 matrix, and a gap weight of 16, 14, 12, 10, 8, 6, or 4 and a length weight of 1, 2, 3, 4, 5, or 6. These different parameters will yield slightly different results but the overall percentage identity of two sequences is not significantly altered when using different algorithms.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

“Sequence identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95%, e.g., at least 96%, 97%, 98%, 99%, or 100% “sequence identity” to a reference nucleotide sequence, it is intended that the nucleotide sequence of the given polynucleotide is identical to the reference sequence except that the given polynucleotide sequence may include up to 5, 4, 3, 2, 1, or 0 point mutations per each 100 nucleotides of the reference nucleotide sequence. In other words, in a polynucleotide having a nucleotide sequence having at least 95%, e.g., at least 96%, 97%, 98%, 99%, or 100% sequence identity relative to the reference nucleotide sequence, up to 5%, 4%, 3%, 2%, 1%, or 0% of the nucleotides in the reference sequence may be deleted or substituted with another nucleotide, or a number of nucleotides up to 5%, 4%, 3%, 2%, 1%, or 0% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95%, e.g., at least 96%, 97%, 98%, 99%, or 100% sequence identity to a reference amino acid sequence, it is intended that the given amino acid sequence of the polypeptide is identical to the reference sequence except that the given polypeptide sequence may include up to 5, 4, 3, 2, 1, or 0 amino acid alterations per each 100 amino acids of the reference amino acid sequence. In other words, to obtain a given polypeptide sequence having at least 95%, e.g., at least 96%, 97%, 98%, 99%, or 100% sequence identity with a reference amino acid sequence, up to 5%, 4%, 3%, 2%, 1%, or 0% of the amino acid residues in the reference sequence may be deleted or substituted with another amino acid, or a number of amino acids up to 5%, 4%, 3%, 2%, 1%, or 0% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

As used herein, the term “immune response” includes innate immune responses, T-cell mediated immune responses, and/or B-cell mediated immune responses. Exemplary immune responses include T cell responses, e.g., cytokine production and cellular cytotoxicity, and B cell responses, e.g., antibody production. In addition, the term “immune response” includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes (e.g. neutrophils).

“Parenteral” administration of an immunogenic composition includes, e.g., subcutaneous (s.c.), intravenous (i.v.), intramuscular (i.m.), intraperitoneal (i.p.) or intradermal (i.d.) injection, or infusion techniques.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

A “variant” of a polypeptide according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Within the meaning of the present invention, the term “conjoint administration” is used to refer to administration of a composition according to the invention and another therapeutic agent simultaneously in one composition, or simultaneously in different compositions, or sequentially (preferably, within a 24 hour period).

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds.(1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Peptide Compositions of the Invention

Modifications of the amino acid structure of CP1 has led to the discovery of additional peptides that are able to regulate complement activation, such as C1q activity. It was previously demonstrated that modifications such as PEGylation enhanced solubility of the peptides as well as potent inhibition of biological activity compared to the parent molecule (IALILEPICCQERAA; SEQ ID NO: 1) in in vitro assays of classical complement pathway activation/inhibition, myeloperoxidase (MPO) inhibition, antioxidant activity and inhibition of NET activity. A peptide with a C-terminal monodisperse 24-mer PEGylated moiety was found to be highly soluble and had strong inhibition of the complement system (IALILEPICCQERAA-dPEG24; SEQ ID NO: 2; PA-DPEG24; PA-dPEG24; RLS-0071). A sarcosine substitution scan of SEQ ID NO: 2 revealed that replacement of isoleucine at position 8 with sarcosine resulted in a peptide, IALILEP(Sar)CCQERAA (PA-I8Sar; RLS-0088; SEQ ID NO: 3) that was water soluble without PEGylation (as described in U.S. Pat. No. 10,005,818).

The term “peptide(s),” as used herein, refers to amino acid sequences, which may be naturally occurring, or peptide mimetics, peptide analogs and/or synthetic derivatives (including for example but not limitation PEGylated peptides) of about 15 amino acids based on SEQ ID NO: 2 and/or 3. In addition, the peptide may be less than about 15 amino acid residues, such as between about 10 and about 15 amino acid residues and such as peptides between about 5 to about 10 amino acid residues. Peptide residues of, for example, 5, 6, 7, 8, 9, 10, 11, 12, 13, 14, and 15 amino acids are equally likely to be peptides within the context of the present invention. Peptides can also be more than 15 amino acids, such as, for example, 16, 17, 18, 19, and 20, or more amino acids.

The disclosed peptides are generally constrained (that is, have some element of structure as, for example, the presence of amino acids that initiate a R turn or R pleated sheet, or, for example, are cyclized by the presence of disulfide bonded Cys residues) or unconstrained (that is, linear) amino acid sequences of greater than about 15 amino acid residues, about 15 amino acid residues, or less than about 15 amino acid residues.

Substitutes for an amino acid within the peptide sequence may be selected from other members of the class to which the amino acid belongs. For example, the nonpolar (hydrophobic) amino acids include alanine, leucine, isoleucine, valine, proline, phenylalanine, tryptophan, and methionine. Amino acids containing aromatic ring structures include phenylalanine, tryptophan, and tyrosine. The polar neutral amino acids include glycine, serine, threonine, cysteine, tyrosine, asparagine, and glutamine. The positively charged (basic) amino acids include arginine and lysine. The negatively charged (acidic) amino acids include aspartic acid and glutamic acid. For example, one or more amino acid residues within the sequence can be substituted by another amino acid of a similar polarity, which acts as a functional equivalent, resulting in a silent alteration.

A conservative change generally leads to less change in the structure and function of the resulting protein. A non-conservative change is more likely to alter the structure, activity, or function of the resulting protein. For example, the peptide of the present disclosure comprises one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

Particularly preferred amino acid substitutions include:
a) Ala for Glu or vice versa, such that a negative charge may be reduced;
b) Lys for Arg or vice versa, such that a positive charge may be maintained;
c) Ala for Arg or vice versa, such that a positive charge may be reduced;
d) Glu for Asp or vice versa, such that a negative charge may be maintained;
e) Ser for Thr or vice versa, such that a free —OH can be maintained;
f) Gln for Asn or vice versa, such that a free NH2 can be maintained;
g) Ile for Leu or for Val or vice versa, as roughly equivalent hydrophobic amino acids;
h) Phe for Tyr or vice versa, as roughly equivalent aromatic amino acids; and
i) Ala for Cys or vice versa, such that disulfide bonding is affected.

Substitutes for an amino acid within the peptide sequence may be selected from any amino acids, including, but not limited to alanine, arginine, asparagine, aspartic acid, cysteine, glutamic acid, glutamine, glycine, histidine, isoleucine, leucine, lysine, methionine, phenylalanine, proline, pyrolysine, selenocysteine, serine, threonine, tryptophan, tyrosine, valine, N-formyl-L-methionine, sarcosine, or other N-methylated amino acids. In some embodiments, sarcosine substitutes for an amino acid within the peptide sequence.

In one embodiment, the invention discloses synthetic peptides derived from human astrovirus coat protein, the peptides comprising the amino acid sequences and modifications of SEQ ID NO: 2 and/or 3.

TABLE 1
List of Peptides of the Invention.

| SEQ | | |
|---|---|---|
| ID | | |
| NO. | Sequence | Description |
| 1 | IALILEPICCQERAA | PA (PIC1) |
| 2 | IALILEPICCQERAA-PEG24 | PA-dPEG24; RLS-0071 |
| 3 | IALILEP(Sar)CCQERAA | PA-18Sar; RLS-0088 |

In other embodiments, the synthetic peptides are capable of altering cytokine expression, including but not limited to models of acute lung injury (ALI). In some embodiments, the invention provides a method of altering cytokine expression comprising administering to the subject in need thereof a composition comprising a therapeutically effective amount of a synthetic peptide comprising SEQ ID NO: 2 and/or 3.

In other embodiments, the synthetic peptides are capable of inhibiting or altering neutrophil binding and/or adhesion. In some embodiments, the invention provides a method of inhibiting or altering neutrophil binding and/or adhesion comprising administering to the subject in need thereof a composition comprising a therapeutically effective amount of a synthetic peptide comprising SEQ ID NO: 2 and/or 3.

In other embodiments, the synthetic peptides are capable of improving neutrophil survival. In some embodiments, the invention provides a method of improving neutrophil survival comprising administering to the subject in need thereof a composition comprising a therapeutically effective amount of a synthetic peptide comprising SEQ ID NO:2 and/or 3.

In other embodiments, the synthetic peptides can bind cell surface receptors such as for example but not limitation, integrin and/or ICAMs, in vivo. In some embodiments, the method provides a method of inhibiting or altering neutrophil binding to cell surface receptors comprising administering to the subject in need thereof a composition comprising a therapeutically effective amount of a synthetic peptide comprising SEQ ID NO: 2 and/or 3.

The disclosed peptides can selectively regulate C1q and MBL activation without affecting alternative pathway activity and are, thus, ideal for preventing and treating diseases mediated by the dysregulated activation of the classical and lectin pathways. Specific blockade of classical and lectin pathways are particularly needed, as both of these pathways have been implicated in ischemia-reperfusion induced injury in many animal models. [Castellano et al., “Therapeutic targeting of classical and lectin pathways of complement protects from ischemia-reperfusion-induced renal damage.” Am J Pathol. 2010; 176(4):1648-59; Lee et al., “Early complement factors in the local tissue immunocomplex generated during intestinal ischemia/reperfusion injury.” Mol. Immunol. 2010 February; 47(5):972-81; Tjernberg, et al., “Acute antibody-mediated complement activation mediates lysis of pancreatic islets cells and may cause tissue loss in clinical islet transplantation.” Transplantation. 2008 Apr. 27; 85(8):1193-9; Zhang et al. “The role of natural IgM in myocardial ischemia-reperfusion injury.” J Mol Cell Cardiol. 2006 July; 41(1):62-7). The alternative pathway is essential for immune surveillance against invading pathogens, and humans with alternative pathway defects suffer severe bacterial infections. By binding and inactivating C1q and MBL, the peptides can efficiently regulate classical and lectin pathway activation while leaving the alternative pathway intact.

The term “regulate,” as used herein, refers to i) controlling, reducing, inhibiting or regulating the biological function of an enzyme, protein, peptide, factor, byproduct, or derivative thereof, either individually or in complexes; ii) reducing the quantity of a biological protein, peptide, or derivative thereof, either in vivo or in vitro; or iii) interrupting a biological chain of events, cascade, or pathway known to comprise a related series of biological or chemical reactions. The term “regulate” may thus be used, for example, to describe reducing the quantity of a single component of the complement cascade compared to a control sample, reducing the rate or total amount of formation of a component or complex of components, or reducing the overall activity of a complex process or series of biological reactions, leading to such outcomes as cell lysis, formation of convertase enzymes, formation of complement-derived membrane attack complexes, inflammation, or inflammatory disease. In an in vitro assay, the term “regulate” may refer to the measurable change or reduction of some biological or chemical event, but the person of ordinary skill in the art will appreciate that the measurable change or reduction need not be total to be “regulatory.”

In any embodiment, the present invention relates to therapeutically active peptides having the effects of regulating the complement system and of preventing, treating and/or mitigating toxic side effects of checkpoint inhibitors, such as intestinal necrosis or damage.

In any embodiment, the present invention relates to therapeutically active peptides having the effect of preventing, treating and/or mitigating intestinal necrosis or damage, e.g., necrosis or damage resulting from severe inflammatory responses, intestinal infarction (i.e., ischemia reperfusion injury of intestinal tissue), autoimmune inflammatory bowel disease (IBD) and associated therapies, and chemotherapy-induced or toxin-induced intestinal necrosis.

### Pharmaceutical Compositions of the Invention

The present disclosure provides pharmaceutical compositions capable of regulating the complement system, comprising at least one peptide, as discussed above, and at least one pharmaceutically acceptable carrier, diluent, stabilizer, or excipient. Pharmaceutically acceptable carriers, excipients, or stabilizers are nontoxic to recipients at the dosages and concentrations employed. They can be solid, semi-solid, or liquid. The pharmaceutical compositions of the present invention can be in the form of tablets, pills, powders, lozenges, sachets, cachets, elixirs, suspensions, emulsions, solutions, or syrups.

The pharmaceutical compositions of the present invention are prepared by mixing the peptide having the appropriate degree of purity with pharmaceutically acceptable carriers, diluents, or excipients. Examples of formulations and methods for preparing such formulations are well known in the art. The pharmaceutical compositions of the present invention are useful as a prophylactic and therapeutic agent for various disorders and diseases, as set forth above. In one embodiment, the composition comprises a therapeutically effective amount of the peptide. In another embodiment, the composition comprises at least one other active ingredient effective in regulating the complement system. In another embodiment, the composition comprises at least one other active ingredient effective in treating at least one disease associated with the complement system. In another embodiment, the composition comprises at least one other active ingredient effective in treating at least one disease that is not associated with the complement system. The term “therapeutically effective amount,” as used herein, refers to the total amount of each active component that is sufficient to show a benefit to the subject.

The therapeutically effective amount of the peptide varies depending on several factors, such as the condition being treated, the severity of the condition, the time of administration, the route of administration, the rate of excretion of the peptide employed, the duration of treatment, the co-therapy involved, and the age, gender, weight, and condition of the subject, etc. One of ordinary skill in the art can determine the therapeutically effective amount. Accordingly, one of ordinary skill in the art may need to titer the dosage and modify the route of administration to obtain the maximal therapeutic effect.

The effective daily dose generally is within the range of from about 0.001 to about 200 milligrams per kilogram (mg/kg) of body weight, including about 5 to about 160 mg/kg, about 10 to about 160 mg/kg, about 40 mg/kg to about 160 mg/kg, and about 40 mg/kg to about 100 mg/kg. This dose can be achieved through a 1-6 time(s) daily dosing regimen. Alternatively, optimal treatment can be achieved through a sustained release formulation with a less frequent dosing regimen. In some embodiments, the therapeutically effective amount of SEQ ID NO: 2 and/or 3 is about 10 mg/kg to about 160 mg/kg. In some embodiments, the therapeutically effective amount of SEQ ID NO: 2 and/or 3 is about 20 mg/kg to about 160 mg/kg. In some embodiments, the therapeutically effective amount of SEQ ID NO: 2 and/or 3 is about 40 mg/kg to about 160 mg/kg. In some embodiments, the therapeutically effective amount of SEQ ID NO: 2 and/or 3 is administered in at least one dose, the first dose comprising about 1 mg/kg to about 160 mg/kg SEQ ID NO: 2 and/or 3. In some embodiments, a second dose comprising a therapeutically effective amount of SEQ ID NO: 2 and/or 3 is administered, the second dose comprising about 1 mg/kg to about 120 mg/kg SEQ ID NO: 2 and/or 3, including about 10 mg/kg to about 120 mg/kg SEQ ID NO: 2 and/or 3. In some embodiments, the therapeutically effective amount of SEQ ID NO: 2 and/or 3 is administered in two doses, the first dose comprising about 1 mg/kg to about 160 mg/kg SEQ ID NO: 2 and/or 3, including about 10 mg/kg to about 120 mg/kg SEQ ID NO: 2 and/or 3, and the second dose comprising about 1 mg/kg to about 120 mg/kg SEQ ID NO: 2 and/or 3, including about 1 mg/kg to about 40 mg/kg SEQ ID NO: 2 and/or 3. In some embodiments, a second dose is administered 30 seconds to 10 hours after a first dose is administered, including about eight hours after administration of the first dose. In some embodiments, the therapeutically effective amount of SEQ ID NO: 2 and/or 3 is administered in multiple doses over a period of about one week to about two weeks, each dose comprising about 1 mg/kg to about 160 mg/kg SEQ ID NO: 2 and/or 3, including about 1 mg/kg to about 120 mg/kg SEQ ID NO: 2 and/or 3, being administered every 4 to 10 hours, including about every eight hours.

In some embodiments, at least one loading dose of about 10 mg/kg to about 160 mg/kg SEQ ID NO: 2 and/or 3 is administered, including about 10 mg/kg to about 120 mg/kg SEQ ID NO: 2 and/or 3, followed by at least one maintenance dose of about 1 mg/kg to about 120 mg/kg SEQ ID NO: 2 and/or 3, including about 1 mg/kg to about 40 mg/kg SEQ ID NO: 2 and/or 3. In some embodiments, the first maintenance dose is administered 4 to 10 hours after the last loading dose. In some embodiments, the maintenance doses are administered every 4 to 10 hours for a period of about one week to about two weeks. In some embodiments, the first maintenance dose is administered 8 hours after the last loading dose, and the maintenance doses are administered every 8 hours for a period of about one week to about two weeks.

In another aspect, the invention is a pharmaceutical composition comprising a therapeutically effective amount of SEQ ID NO: 2 and/or 3 and at least one pharmaceutically acceptable carrier, diluent, or excipient.

The compositions of the invention can comprise a carrier and/or excipient. While it is possible to use a peptide of the present invention for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice. Oral formulations readily accommodate additional mixtures, such as, e.g., milk, yogurt, and infant formula. Solid dosage forms for oral administration can also be used and can include, e.g., capsules, tablets, caplets, pills, troches, lozenges, powders, and granules. Non-limiting examples of suitable excipients include, e.g., diluents, buffering agents (e.g., sodium bicarbonate), preservatives, stabilizers, binders, compaction agents, lubricants, dispersion enhancers, disintegration agents, antioxidants, flavoring agents, sweeteners, and coloring agents. Those of relevant skill in the art are well able to prepare suitable solutions.

In one embodiment of any of the compositions of the invention, the composition is formulated for delivery by a route such as, e.g., oral, topical, rectal, mucosal, sublingual, nasal, naso/oro-gastric gavage, parenteral, intraperitoneal, intradermal, transdermal, intrathecal, nasal, and intratracheal administration. In one embodiment of any of the compositions of the invention, the composition is in a form of a liquid, foam, cream, spray, powder, or gel. In one embodiment of any of the compositions of the invention, the composition comprises a buffering agent (e.g., sodium bicarbonate).

Administration of the peptides and compositions in the methods of the invention can be accomplished by any method known in the art. Non-limiting examples of useful routes of delivery include oral, rectal, fecal (by enema), and via naso/oro-gastric gavage, as well as parenteral, intraperitoneal, intradermal, transdermal, intrathecal, nasal, and intratracheal administration. The active agent may be systemic after administration or may be localized by the use of regional administration, intramural administration, or use of an implant that acts to retain the active dose at the site of implantation.

The useful dosages of the compounds and formulations of the invention can vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses may be effective to achieve a therapeutic effect. While it is possible to use a compound of the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

Solutions or suspensions can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity, such as sodium chloride or dextrose.

In instances in which the agents exhibit insufficient solubility, methods for solubilizing agents may be used. Such methods are known to those of skill in this art, and include, but are not limited to, using co-solvents, such as, e.g., dimethylsulfoxide (DMSO), using surfactants, such as TWEEN®80, or dissolution in aqueous sodium bicarbonate. Pharmaceutically acceptable derivatives of the agents may also be used in formulating effective pharmaceutical compositions.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art (e.g., Remington's Pharmaceutical Sciences, Mack Publishing Company, Easton, Pa., 15th Edition, 1975). The composition or formulation to be administered will, in any event, contain a quantity of the active agent in an amount sufficient to alleviate the symptoms of the treated subject.

The active agents or pharmaceutically acceptable derivatives may be prepared with carriers that protect the agent against rapid elimination from the body, such as time release formulations or coatings. The compositions may include other active agents to obtain desired combinations of properties.

Parenteral administration, generally characterized by injection, either subcutaneously, intramuscularly or intravenously, is also contemplated herein. Injectables can be prepared in conventional forms, either as liquid solutions or suspensions, solid forms suitable for solution or suspension in liquid prior to injection, or as emulsions. Suitable excipients include, by way of example and without limitation, water, saline, dextrose, glycerol or ethanol. In addition, if desired, the pharmaceutical compositions to be administered may also contain minor amounts of non-toxic auxiliary substances, such as wetting or emulsifying agents, pH buffering agents, stabilizers, solubility enhancers, and other such agents, such as, for example, sodium acetate, sorbitan monolaurate, triethanolamine oleate and cyclodextrins.

Lyophilized powders can be reconstituted for administration as solutions, emulsions, and other mixtures or formulated as solids or gels. The sterile, lyophilized powder is prepared by dissolving an agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution can be apportioned into vials for lyophilization. Each vial can contain, by way of example and without limitation, a single dosage (10-1000 mg, such as 100-500 mg) or multiple dosages of the agent. The lyophilized powder can be stored under appropriate conditions, such as at about 4° C. to room temperature. Reconstitution of this lyophilized powder with water for injection provides a formulation for use in parenteral administration.

### Combination Therapies

A further embodiment of the invention provides a method of regulating the complement system, comprising administering to a subject a pharmaceutical composition of the present invention. While the pharmaceutical compositions of the present invention can be administered as the sole active pharmaceutical agent, they can also be used in combination with one or more therapeutic or prophylactic agent(s) that is(are) effective for regulating the complement system. In this aspect, the method of the present invention comprises administrating a pharmaceutical composition of the present invention before, concurrently, and/or after one or more additional therapeutic or prophylactic agents effective in regulating the complement system.

The pharmaceutical compositions of the present invention can be administered with additional agent(s) in combination therapy, either jointly or separately, or by combining the pharmaceutical compositions and the additional agent(s) into one composition. The dosage is administered and adjusted to achieve maximal regulation of the complement system. For example, both the pharmaceutical compositions and the additional agent(s) are usually present at dosage levels of between about 10% and about 150%, more preferably, between about 10% and about 80%, of the dosage normally administered in a mono-therapy regimen.

In any embodiment, the pharmaceutical compositions of the invention are administered in combination with a checkpoint inhibitor, either simultaneously or sequentially in any order. For example and not limitation, the pharmaceutical composition can be administered after the checkpoint inhibitor, and/or can be administered as a preventative to a subject who has been treated with a checkpoint inhibitor and has experienced intestinal inflammation, damage, or necrosis as a result of the checkpoint inhibitor. In any embodiment, the pharmaceutical compositions of the invention can be administered to a subject who is currently being treated with a checkpoint inhibitor, has been treated with a checkpoint inhibitor, and/or will be treated with a checkpoint inhibitor. Nonlimiting examples of checkpoint inhibitors include CTLA-4 inhibitors, PD-1 inhibitors and PD-L1 inhibitors, such as pembrolizumab (Keytruda), ipilimumab (Yervoy), nivolumab (Opdivo) and atezolizumab (Tecentriq).

In any embodiment, the pharmaceutical compositions of the invention are capable of preventing, treating and/or mitigating toxic side effects of a checkpoint inhibitor, such as intestinal necrosis and/or damage.

In any embodiment, the pharmaceutical compositions of the invention are capable of preventing, treating and/or mitigating intestinal necrosis and/or damage, e.g., necrosis or damage resulting from severe inflammatory responses, intestinal infarction (i.e., ischemia reperfusion injury of intestinal tissue), autoimmune inflammatory bowel disease (IBD) and associated therapies, and chemotherapy-induced or toxin-induced intestinal necrosis.

### Methods of Use

In an aspect, the present invention provides a method of preventing, treating and/or mitigating toxic side effects of checkpoint inhibitors comprising administering to the subject in need thereof a composition comprising a therapeutically effective amount of a synthetic peptide comprising SEQ ID NO: 2 and/or 3.

In an aspect, the present invention provides a method of preventing, treating and/or mitigating intestinal necrosis and/or damage, e.g., necrosis or damage resulting from severe inflammatory responses, intestinal infarction (i.e., ischemia reperfusion injury of intestinal tissue), autoimmune inflammatory bowel disease (IBD) and associated therapies, and chemotherapy-induced or toxin-induced intestinal necrosis, comprising administering to the subject in need thereof a composition comprising a therapeutically effective amount of a synthetic peptide comprising SEQ ID NO: 2 and/or 3.

In an aspect, the present invention provides a method of preventing, treating and/or mitigating intestinal necrosis and/or damage in a subject who is being treated, has been treated, and/or will be treated with at least one checkpoint inhibitor comprising administering to the subject in need thereof a composition comprising a therapeutically effective amount of a synthetic peptide comprising SEQ ID NO: 2 and/or 3.

In any embodiment of any of the foregoing methods, the composition further comprises at least one pharmaceutically acceptable carrier, diluent, stabilizer, or excipient. In an embodiment of any of the foregoing methods, the therapeutically effective amount of SEQ ID NO: 2 and/or 3 is about 10 mg/kg to about 160 mg/kg. In an embodiment of any of the foregoing methods, the therapeutically effective amount of SEQ ID NO: 2 and/or 3 is about 20 mg/kg to about 160 mg/kg. In an embodiment of any of the foregoing methods, the therapeutically effective amount of SEQ ID NO: 2 and/or 3 is about 40 mg/kg to about 160 mg/kg. In an embodiment of any of the foregoing methods, the composition is formulated for subcutaneous, intravenous, intraperitoneal, or intramuscular administration. In an embodiment, the composition further comprises a pharmaceutically acceptable carrier and/or excipient.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Use of PA-dPEG24 to Prevent, Treat and/or Mitigate Intestinal Necrosis

Intestinal necrosis is a potentially life-threatening medical condition that can arise from a variety of clinical etiologies. The necrosis of intestinal tissue leads to compromise of the lumen and leakage of enteric bacteria as well as precipitation of aggressive immunological responses including the complement system and neutrophils. PA-dPEG24 (RLS-0071) is a peptide inhibitor of the classical and lectin pathways as well as myeloperoxidase activity and NETosis, which are major effectors of neutrophils. Therefore, the inventors decided to evaluate the extent to which immunomodulation via inhibition of the complement system and neutrophil effectors could affect survival in the setting of intestinal necrosis and leakage of intestinal contents. Adolescent male Long-Evans rats were subject to cecal ligation and puncture (CLP), an established rat model of intestinal necrosis, with one cohort receiving a single dose of 40 mg/kg RLS-0071 thirty minutes after surgery while the control group received no treatment. Survival of the rats was then assessed up to 5 days after surgery. Surprisingly, animals treated with RLS-0071 demonstrated 80% survival compared to 50% for the untreated group. In order to evaluate whether the unexpected increase in survival could be due to moderation of inflammatory responses, the inventors assessed markers of NETosis, free DNA in plasma, and the pro-inflammatory cytokine IL-6. A reduction in blood levels of free DNA and the inflammatory cytokine IL-6 were observed for animals treated with RLS-0071. These results demonstrate that a single dose of RLS-0071 can increase survival of intestinal necrosis, perhaps by reducing certain inflammatory responses.

### Materials and Methods

### Animal Experiments

The previously established CLP model [10] was utilized with modifications. Male Long-Evans rats (250-300 grams) were induced using 5% isoflurane with oxygen and anesthesia was maintained by isoflurane delivered via nose cone at 1.5-2% with oxygen. Adequate anesthesia was confirmed with a toe pinch. The surgical area (lower abdominal quadrants) was shaved, cleaned with 10% iodine and subsequently cleaned with 70% ethanol for a total of three times. For pain management, buprenorphine-SR (1 mg/kg) was given subcutaneously pre-op. For the surgical procedure, animals were in the supine position with their heads oriented away from the operator. After placing a sterile drape, a longitudinal midline incision (approximately 3-4 cm) was made with a sterile scalpel. After the initial incision, small scissors were used to enter the peritoneal cavity. The linea alba of the abdominal musculature was dissected away for intermuscular incision of fascial and peritoneal layers. The cecum was located and exteriorized, the remainder of the bowel was left in the peritoneal cavity. The mesentery of the cecum was dissected away with care taken to avoid damage to the cecal branch of the ileocecal artery and avoid bleeding complications. The cecum was ligated at 75% using sterile 3-0 non-absorbable suture. The cecum was perforated twice by through-and-through punctures using a sterile 18-gauge needle midway between the ligation and the tip of the cecum in a mesenteric-to-antimesenteric direction. The cecum was relocated into the abdominal cavity. The peritoneum was closed using 4-0 non-absorbable sutures and the skin was closed with sterile metallic wound clips. The animals received 5 mL of prewarmed (37° C.) normal saline subcutaneously for recovery. For RLS-0071 treated animals (n=9), the rats received a single dose of 40 mg/kg RLS-0071 30 minutes after surgery intravenously through the indwelling jugular catheter. The peptide, consisting of the amino acid sequence IALILEPICCQERAA (SEQ ID NO: 2) with a monodisperse, 24mer polyethylene glycol (PEG) tail, was manufactured by PolyPeptide Group (San Diego, CA) to >95% purity as verified by HPLC and mass spectrometry analysis. Lyophilized RLS-0071 was solubilized in 0.05 M Histidine buffer and pH adjusted to 6.5. The control group (n=6) did not receive compound. Animals were monitored every 30 minutes for at least two hours post-surgery. Once bright, alert, and responsive, the animals were placed back into their home cage. A quantitative assessment of morbidity was conducted on the animals twice a day for the duration of the study. (Shrum, B., Anantha, R. V., Xu, S. X. et al. A robust scoring system to evaluate sepsis severity in an animal model. BMC Res Notes 7, 233 (2014). https://doi.org/10.1186/1756-0500-7-233). Scoring criteria included appearance, level of consciousness, activity, response to stimulus, eyes open vs. closed, respiration rate and respiration quality. Each category was ranked 0 (best)-4 (worst). If an animal's cumulative score was greater than 21 or if respiration quality was great than 3, the animal was humanely euthanized. At the end of 5 days, surviving animals, were euthanized by carbon dioxide asphyxiation and cervical dislocation. The animals were subsequently monitored at a minimum of twice/day.

Blood samples were collected before surgery (pre-bleed) and 24 hours after surgery. Plasma was isolated from the blood and analyzed for free DNA concentration and cytokines levels as described below.

### Plasma DNA Measurements

Free DNA was measured by PicoGreen in rat plasma samples as previously described [8] Briefly, plasma samples were diluted in 10 mM Tris-HCl, 1 mM EDTA, pH 8.0 (TE) buffer and 50 uL of each sample was added to the wells along with 50 uL of a 1:200 dilution of PicoGreen (Life Technologies, Carlsbad, CA, USA) and incubated at room temperature for 10 minutes, protected from light. A DNA standard curve was prepared in TE buffer. The fluorescence was then read at an excitation wavelength of 485 nm and an emission wavelength of 520 nm using a BioTek microplate reader. All free DNA measurements were done in triplicate.

### Cytokine Analysis

The Rat IL-6 ELISA was purchased from R&D Systems. Plasma samples from experimental animals were run according to the manufacturer's instructions. Briefly, diluted rat plasma samples were added to wells pre-coated with capture IL-6 antibodies overnight. The plate was blocked for 60 min at room temperature with 1% BSA. 100 uL of plasma samples (diluted 1:4) or standards was added to the plate and incubated at room temperature for 2 hrs, then any unbound components were removed by washing. Next, 100 μL of detection IL-6 antibody (diluted 1:60) was added and incubated for 2 hrs at room temperature and washed. Next, 100 μL of streptavidin-horseradish peroxidase (HIRP) conjugate (diluted 1:40) was added and incubated in the dark for 20 minutes at room temperature. Following washing, 100 μL of 3,3′,5,5′-tetramethylbenzidine (TMB) substrate was added for approximately 5 minutes at room temperature. The reaction was stopped with the addition of 100 μL of sulfuric acid and absorbance measured at 450 nm using a BioTek microplate reader. Sample concentrations were calculated using an IL-6 standard curve made with 1:2 serial dilutions.

### Statistical Analysis

Data are represented as mean and standard error of the mean.

### Results

### RLS-0071 Reduces Mortality in CLP Model

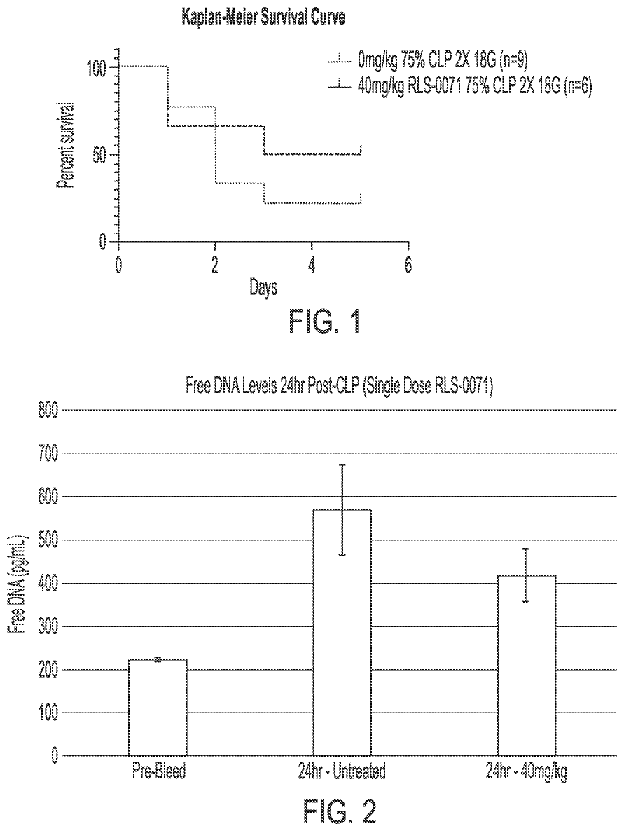

In order to test whether modulation of the complement system and neutrophil immune mechanisms would increase death due to bacterial sepsis in the setting of intestinal necrosis, the inventors tested RLS-0071 in the CLP model. In the CLP model, the ligation of the cecum causes infarction of the cecal punch, which then undergoes necrosis. The previously developed CLP model in Long-Evans rats reported that induction of a mid-grade necrosis resulting in survival rates of ˜40% required the cecum to be ligated at half the distance between distal pole and the base of the cecum followed by through and through puncture of the ligated cecum with an 18-gauge needle [11]. The inventors' experimental protocol utilized a 75% ligation of the cecum followed by two, 18-gauge needle punctures to the cecum, resulting in 20% survival. Surprisingly, compared to the no-rescue treatment group, rats receiving CLP and a single dose of 40 mg/kg RLS-0071 delivered intravenously 30 minutes after surgery experienced 50% survival, a 2.5-fold increase as shown in FIG. 1. Thus, rather than modulation of the complement system and neutrophil effectors causing an increase in death due to overwhelming infection, administration of RLS-0071 appeared to increase survival in the setting of intestinal necrosis.

### RLS-0071 Inhibits Free DNA Accumulation in the Blood

The finding that RLS-0071 may have increased survival in the setting of intestinal necrosis raised the possibility of whether the inhibition of inflammation in the rats was affecting their survival. The inventors then assayed for two key aspects of inflammation in the blood of CLP animals. Neutrophil extracellular traps (NETs) released from activated neutrophils have been previously shown to play a pathogenic role in a variety of autoimmune, metabolic and inflammatory diseases [12] and are hypothesized to contribute to immunothrombosis and disseminated intravascular coagulation (DIC) in sepsis [4]. NETs have been observed in murine models of virally induced acute lung injury. The second aspect, the presence of free DNA in the bloodstream, is a biomarker for NETs in the blood of human patients with acute lung injury [13,14] as well as COVID-19 patients [15]. To ascertain if free DNA was present in the blood of septic rats, the inventors measured levels of free DNA by PicoGreen fluorescence 24 hours post-surgery. Compared to blood taken from the animals before surgery (pre-bleed), free DNA levels were increased in animals subject to the CLP procedure. Animals receiving RLS-0071 had a reduction in the level of free DNA compared to animals not receiving treatment. Further, the animals were tested for IL-1B and found to be negative. This reduction in free DNA in animals treated with RLS-0071 suggests that RLS-0071 reduces NET formation in this model as shown in FIG. 2.

### RLS-0071 Reduces Inflammatory Cytokine IL-6 Levels in the Blood

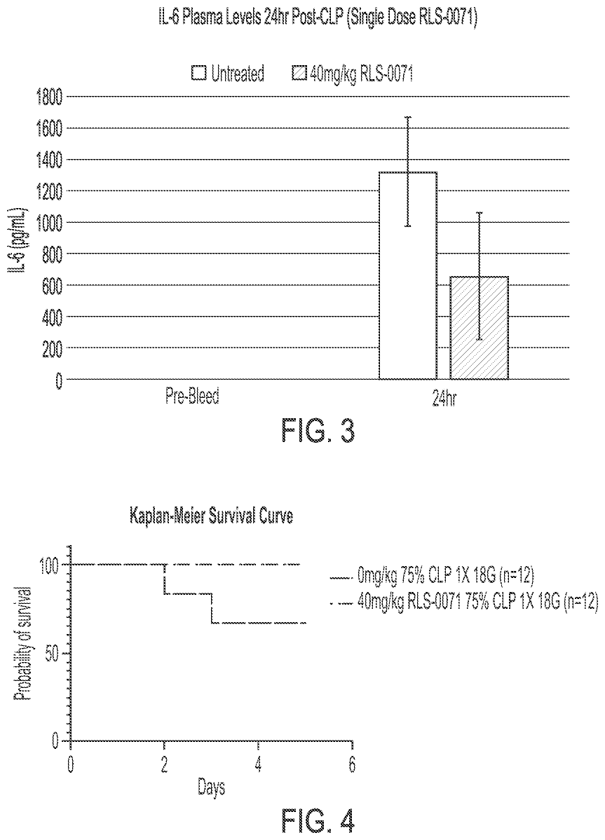

In cases of intestinal necrosis, significant amounts of pro-inflammatory cytokines are generated in response to infection. This so-called ‘cytokine storm’ has been well documented for sepsis and this aggressive inflammatory response is associated with severe outcomes including end-organ damage and sometimes death [4]. To ascertain the effect of RLS-0071 on inflammatory cytokine levels in this model, levels of IL-6 from the blood were measured. IL-6 is a powerful pro-inflammatory cytokine released predominantly by macrophages and plays a major role in many inflammatory diseases including inflammatory bowel diseases [16]. Blood taken from the animals before surgery (pre-bleed) had no detectable levels of IL-6. Animals subject to the CLP procedure demonstrated an increase in the blood levels of the cytokine at 24 hours post-surgery. In contrast, animals receiving RLS-0071 showed a reduction in IL-6 compared with no-rescue-intervention animals, suggesting that RLS-0071 can reduce inflammatory cytokine production in this model as shown in FIG. 3. Further, the animals were tested for IL-1B and found to be negative.

### Discussion

The inventors conducted experiments to determine if the immunomodulatory molecule RLS-0071 could affect survival from bacterial sepsis in the setting of intestinal necrosis. Surprisingly, RLS-0071 was able to increase survival in an established model of intestinal necrosis resulting from cecal ligation. As previously reported, the cecal ligation and puncture (CLP) rat model has been utilized for over 40 years and is considered a gold standard as a model for intestinal necrosis and sepsis [11]. In this model, the cecum is ligated below the ileocecal valve followed by needle puncture of the cecum. Upon necrosis and perforation of the cecum, bacteria, toxins and other microbial contaminants are released into the peritoneal cavity, resulting in bacterial peritonitis. Subsequently, these mixed enteric bacteria are transported into the blood compartment causing bacterial sepsis. Enteric pathogen bacterial sepsis commonly precipitates hypotension, disseminated intravascular coagulation (DIC), multisystem organ failure, and potentially death.

RLS-0071 has been shown to inhibit classical complement activation in in vitro, in vivo and ex vivo studies and to inhibit NET formation via inhibition of myeloperoxidase in in vitro and ex vivo studies [6-8]. Given the immunomodulatory activities upon the complement system and neutrophil effectors, the inventors hypothesized that RLS-0071 could worsen the bacterial sepsis and increase fatality in the CLP animal model. Our results demonstrated the surprising finding that RLS-0071 delivered as a single dose 30 minutes post-surgery increased survival by 2.5-fold. This result suggested that RLS-0071 may be increasing survival by decreasing vital aspects of the inflammatory response to intestinal necrosis. CLP animals treated with RLS-0071 showed decreased levels of free DNA, which serves as a biomarker for NETosis, and reduction of the inflammatory cytokine IL-6. The ability of RLS-0071 to increase survival and reduce inflammation in the CLP model has potential for utility as a clinical therapeutic for various disease processes associated with intestinal necrosis such as intestinal infarction (i.e., ischemia reperfusion injury of intestinal tissue), autoimmune inflammatory bowel disease (IBD) and associated medication, chemotherapy-induced or toxin-induced intestinal necrosis, and intestinal necrosis or damage resulting from severe inflammatory responses.

### Example 2: Multi-Dose Administration of RLS-0071 and RLS-0088 Reduces Mortality in CLP Model

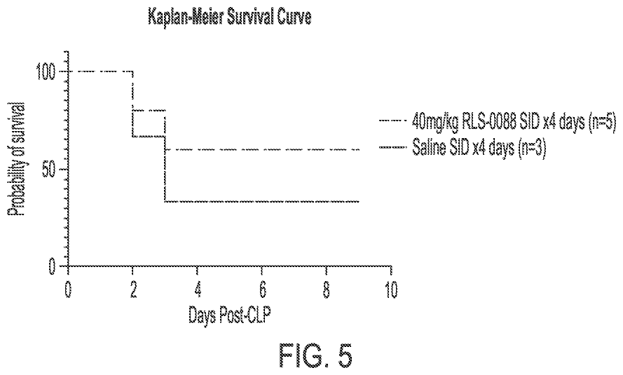

The inventors next assessed if a multi-dose regimen of RLS-0071 would have an effect on rate of survival. RLS-0071 was administered to the animals as in Example 1 at 0.5, 24, 48 and 72 hours after surgery. The rats receiving RLS-0071 showed a significant increase in survival (p=0.032) (FIG. 4). The second generation EPICC peptide, RLS-0088, was then tested in the CLP model to determine if it had similar effects on improving survival. Using the same multidose experimental approach as set out in Example 1, rats were subjected to CLP and 40 mg/kg RLS-0088 was administered at 0.5, 24, 48 and 72 hours after surgery. The rats receiving RLS-0088 demonstrated an increase in survival (FIG. 5).

### Example 3: Administration of Pharmaceutical Formulations

A pharmaceutical composition comprising a therapeutically effective amount of SEQ ID NO: 2 and/or 3 is administered to a subject in need thereof to treat a disease or condition. The administration can be by any appropriate route (e.g., injection, infusion, implantation, intravenous administration, subcutaneous administration, intraperitoneal administration, intramuscular administration).

A pharmaceutical composition comprising a therapeutically effective amount of SEQ ID NO: 2 and/or 3 is administered to a subject in need thereof to regulate the complement system in the subject. The administration can be by any appropriate route (e.g., injection, infusion, implantation, intravenous administration, subcutaneous administration, intraperitoneal administration, intramuscular administration).

A pharmaceutical composition comprising a therapeutically effective amount of SEQ ID NO: 2 and/or 3 is administered to a subject in need thereof to alter cytokine expression in the subject. The administration can be by any appropriate route (e.g., injection, infusion, implantation, intravenous administration, subcutaneous administration, intraperitoneal administration, intramuscular administration).

A pharmaceutical composition comprising a therapeutically effective amount of SEQ ID NO: 2 and/or 3 is administered to a subject in need thereof to prevent, treat and/or mitigate toxic side effects of checkpoint inhibitors, e.g., intestinal necrosis or damage. The administration can be by any appropriate route (e.g., injection, infusion, implantation, intravenous administration, subcutaneous administration, intraperitoneal administration, intramuscular administration).

A pharmaceutical composition comprising a therapeutically effective amount of SEQ ID NO: 2 and/or 3 is administered to a subject in need thereof to prevent, treat and/or mitigate intestinal necrosis and/or damage, e.g., necrosis or damage resulting from severe inflammatory responses, intestinal infarction (i.e., ischemia reperfusion injury of intestinal tissue), autoimmune inflammatory bowel disease (IBD) and associated medication, and chemotherapy-induced or toxin-induced intestinal necrosis. The administration can be by any appropriate route (e.g., injection, infusion, implantation, intravenous administration, subcutaneous administration, intraperitoneal administration, intramuscular administration).

A pharmaceutical composition comprising a therapeutically effective amount of SEQ ID NO: 2 and/or 3 is administered to a subject in need thereof to prevent, treat and/or mitigate intestinal necrosis and/or damage in a subject who is being treated, has been treated, and/or will be treated with at least one checkpoint inhibitor. The administration can be by any appropriate route (e.g., injection, infusion, implantation, intravenous administration, subcutaneous administration, intraperitoneal administration, intramuscular administration).

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

1. Erik A. Campbell, Michael Silberman. Bowel necrosis. Treasure Island (FL): StatPearls Publishing; 2021 January-.
2. Dahiya D S, Wani F, Guidi J C, Kichloo A. Gastrointestinal Adverse Effects of Immunotherapeutic Agents: A Systematic Review. Gastroenterology Res. 2020 December; 13(6):227-232. doi: 10.14740/gr1340. Epub 2020 Dec. 23. PMID: 33447301.
3. Karasu E, Nilsson B, Kohl J, Lambris J D, Huber-Lang M. Targeting Complement Pathways in Polytrauma- and Sepsis-Induced Multiple-Organ Dysfunction. Front Immunol. 2019 Mar. 21; 10:543. doi: 10.3389/fimmu.2019.00543. eCollection 2019. PMID: 30949180
4. Chen Z, Zhang H, Qu M, Nan K, Cao H, Cata J P, Chen W, Miao C. The Emerging Role of Neutrophil Extracellular Traps in Sepsis and Sepsis-Associated Thrombosis. Front Cell Infect Microbiol. 2021 Mar. 17; 11:653228. doi: 10.3389/fcimb.2021.653228.
5. Chakraborty R K, Burns B. Systemic Inflammatory Response Syndrome. 2021 Mar. 1. In: StatPearls [Internet]. Treasure Island (FL): StatPearls Publishing; 2021 January-. PMID: 31613449.
6. Sharp J A, Hair P S, Pallera H K, Kumar P S, Mauriello C T, Nyalwidhe J O, et al. Peptide Inhibitor of Complement C1 (RLS-0071) Rapidly Inhibits Complement Activation after Intravascular Injection in Rats. PLoS One 2015; 10(7):e0132446.
7. Hair P S, Sass L A, Krishna N K, Cunnion K M. Inhibition of Myeloperoxidase Activity in Cystic Fibrosis Sputum by Peptide Inhibitor of Complement C1 (RLS-0071). PLoS One 2017; 12(1):e0170203.
8. Hair P S, Enos Al, Krishna N K, Cunnion K M. Inhibition of Immune Complex Complement Activation and Neutrophil Extracellular Trap Formation by Peptide Inhibitor of Complement C1. Front Immunol 2018 Mar. 26; 9:558.
9. Gregory Rivera M, Hair P S, Cunnion K M, Krishna N K. Peptide Inhibitor of Complement C1 (RLS-0071) demonstrates antioxidant activity via single electron transport (SET) and hydrogen atom transfer (HAT). PLoS One 2018 Mar. 2; 13(3):e0193931.
10. Hair P S, Gregory Rivera M, Enos Al, Pearsall S E, Sharp J A, Yousefieh N, et al. Peptide Inhibitor of Complement C1 (RLS-0071) Inhibits Growth of Pathogenic Bacteria. Int J Pept Res Therap 2017. doi.org/10.1007/s10989-017-9651-z
11. Rittirsch D, Huber-Lang M S, Flierl M A, Ward P A. Immunodesign of experimental sepsis by cecal ligation and puncture. Nat Protoc. 2009; 4(1):31-6. doi: 10.1038/nprot.2008.214. PMID: 19131954.
12. Mutua V and Gershwin U. A review of neutrophil extracellular traps (NETs) in disease: potential anti-NET therapeutics. Clin Rev Allergy Immunol 2020; 1:1-18.
13. Caudrillier A, Kessenbrock K, Gilliss B M, Nguyen J X, Marques M B, Monestier M, et al. Platelets induce neutrophil extracellular traps in transfusion-related acute lung injury. J Clin Invest 2012; 122:2661-2671.
14. Thomas G M, Carbo C, Curtis B R, Martinod K, Mazo I B, Schatzberg D, et al. Extracellular DNA traps are associated with the pathogenesis of TRALI in humans and mice. Blood 2012; 119:6335-6343.
15. Skendros P, Mitsios A, Chrysanthopoulou A, Mastellos D C, Metallidis S, Rafailidis P, et al.

Complement and tissue factor-enriched neutrophil extracellular traps are key drivers in COVID-19 immunothrombosis. J Clin Invest 2020; 130(11):6151-6157.
16. Kamieniak A, Krawiec P, Pac-Kozuchowska E. Interleukin 6: biological significance and role in inflammatory bowel diseases. Pawlowska—Adv Clin Exp Med. 2021 Apr. 28. doi: 10.17219/acem/130356. PMID: 33908198.

1. (canceled)

2. A method of preventing, treating and/or mitigating toxic side effects of checkpoint inhibitors in a subject in need thereof comprising administering to the subject in need thereof a composition comprising a therapeutically effective amount of a synthetic peptide comprising SEQ ID NO: 2 and/or 3.

3. A method of preventing, treating and/or mitigating intestinal necrosis and/or damage in a subject in need thereof comprising administering to the subject in need thereof a composition comprising a therapeutically effective amount of a synthetic peptide comprising SEQ ID NO: 2 and/or 3.

4. A method of preventing, treating and/or mitigating intestinal necrosis and/or damage in a subject who is being treated, has been treated, or will be treated with at least one checkpoint inhibitor comprising administering to the subject in need thereof a composition comprising a therapeutically effective amount of a synthetic peptide comprising SEQ ID NO: 2 and/or 3.

5. The method of claim 2, wherein the composition further comprises at least one pharmaceutically acceptable carrier, diluent, stabilizer, or excipient.

6. The method of claim 2, wherein the therapeutically effective amount of SEQ ID NO: 2 and/or 3 is about 10 mg/kg to about 160 mg/kg.

7. The method of claim 2, wherein the therapeutically effective amount of SEQ ID NO: 2 and/or 3 is about 20 mg/kg to about 160 mg/kg.

8. The method of claim 2, wherein the therapeutically effective amount of SEQ ID NO: 2 and/or 3 is about 40 mg/kg to about 160 mg/kg.

9. The method of claim 2, wherein the therapeutically effective amount of SEQ ID NO: 2 and/or 3 is administered in at least one dose, the first dose comprising about 1 mg/kg to about 160 mg/kg SEQ ID NO: 2 and/or 3.

10. The method of claim 9, wherein a second dose comprising a therapeutically effective amount of SEQ ID NO: 2 and/or 3 is administered, the second dose comprising about 1 mg/kg to about 120 mg/kg SEQ ID NO: 2 and/or 3.

11. The method of claim 2, wherein the therapeutically effective amount of SEQ ID NO: 2 and/or 3 is administered in two doses, the first dose comprising about 1 mg/kg to about 160 mg/kg SEQ ID NO: 2 and/or 3 and the second dose comprising about 1 mg/kg to about 120 mg/kg SEQ ID NO: 2 and/or 3.

12. The method of claim 11, wherein the second dose is administered 30 seconds to 10 hours after the first dose is administered.

13. The method of claim 2, wherein the therapeutically effective amount of SEQ ID NO:2 and/or 3 is administered in multiple doses over a period of about one week to about two weeks, each dose comprising about 1 mg/kg to about 160 mg/kg SEQ ID NO: 2 and/or 3 and being administered every 4 to 10 hours.

14. The method of claim 13, wherein each dose is administered every 8 hours.

15. The method of claim 2, wherein the therapeutically effective amount of SEQ ID NO: 2 and/or 3 is administered in at least one loading dose of about 10 mg/kg to about 160 mg/kg SEQ ID NO: 2 and/or 3, followed by at least one maintenance dose of about 1 mg/kg to about 120 mg/kg SEQ ID NO: 2 and/or 3,
wherein the first maintenance dose is administered 4 to 10 hours after the last loading dose, and
wherein the maintenance doses are administered every 4 to 10 hours for a period of about one week to about two weeks.

16. The method of claim 15, wherein the first maintenance dose is administered 8 hours after the last loading dose, and wherein the maintenance doses are administered every 8 hours for a period of about one week to about two weeks.

17. The method of claim 2, wherein the subject is also being treated with a checkpoint inhibitor, has previously been treated with a checkpoint inhibitor, or will be treated with a checkpoint inhibitor.

18. The method of claim 17, wherein the checkpoint inhibitor is selected from the group consisting of CTLA-4 inhibitors, PD-1 inhibitors and PD-L1 inhibitors, such as pembrolizumab (Keytruda), ipilimumab (Yervoy), nivolumab (Opdivo) and atezolizumab (Tecentriq).


SEQUENCE LISTING (3 sequences)

SEQ ID NO: 1 - protein, 15 aa, Artificial Sequence
  Description of Artificial Sequence Synthetic peptide
IALILEPICCQERAA

SEQ ID NO: 2 - protein, 15 aa, Artificial Sequence
  Description of Artificial Sequence Synthetic peptide
  C-term dPEG24
IALILEPICCQERAA

SEQ ID NO: 3 - protein, 15 aa, Artificial Sequence
  Description of Artificial Sequence Synthetic peptide
  MOD_RES (8)..(8): Sarcosine
IALILEPXCCQERAA